(19) United States
(12) Patent Application Publication
(10) Pub. No.: US 2004/0253140 A1
(43) Pub. Date: Dec. 16, 2004

(54) Method and kit for mechanically cleaning and sterilizing medical instruments

(76) Inventors: Wolfgang Wagemann, Tremsbuttel (DE); Markus Kamer, Reinbek (DE)

Correspondence Address: Medlen & Carroll, 101 Howard Street, Suite 250, San Francisco, CA, 94105, US

(21) Appl. No.: 10/494,871

(22) PCT Filed: Oct. 11, 2002
(86) PCT No.: PCT/EP02/11421

(30) Foreign Application Priority Data
Nov. 9, 2001 (EP) 01126781.2

(51) Int. Cl. A61L 2/18

(52) U.S. Cl. 422/29; 422/37

(57) ABSTRACT

The invention concerns a method for mechanically cleaning and sterilizing medical and surgical instruments and appliances, which consists in: (a) cleaning the instruments and appliances with an aqueous cleaning solution and (b) sterilizing the instruments and appliances with an aqueous sterilizing solution. The invention is characterized in that chlorine dioxide is released in situ, from chlorite and an acid activator, as disinfectant during the sterilizing step. The invention is based on the use of chlorine dioxide as efficient disinfectant, not requiring separate production of said substance in a chlorine dioxide generator. The inventive method can be implemented in a standard instrument washing machine.

The invention relates to a method for mechanically cleaning and disinfecting medical and surgical instruments and apparatuses, for example endoscopes and/or parts thereof, having the following steps:.

a) cleaning the instruments and apparatuses with an aqueous cleaning solution,

b) disinfecting the instruments and apparatuses with an aqueous disinfectant solution.

The invention further relates to a kit for carrying out the method.

In medical therapy and diagnostics, a multiplicity of interventions are carried out using endoscopes. These endoscopes are soiled during their use and are infected with many types of microorganisms.

Cleaning and disinfecting used endoscopes is difficult, since endoscopes, in particular modern glass-fiber endoscopes, have surfaces made of a multiplicity of different materials which are in part temperature-sensitive and in part susceptible to corrosion. A problem is also the complete cleaning and disinfection of the multiplicity of cavities in endoscopes, in particular the narrow-bore channels present in the interior.

A method mentioned at the outset is disclosed by EP-A-0 268 227. The aqueous disinfectant solution comprises glutaraldehyde as disinfectant. Many aldehydes are hazardous to health and can only be handled using special precautionary measures.

WO-A-96/10916 discloses disinfecting medical instruments with chlorine dioxide. There, a stock solution is made up from a sodium chlorite solution on the one hand and an acid on the other, which stock solution is used for the disinfection after dilution.

The object underlying the invention is to provide a method of the type mentioned at the outset using which simple, rapid and effective mechanical cleaning and disinfection of medical instruments and apparatuses is possible.

The inventive method is characterized in that, during the disinfection step, as disinfectant, chlorine dioxide is released in situ from a chlorite (for example an alkali metal chlorite such as sodium chlorite).

First, let some terms used in the context of the invention be explained.

Medical/surgical instruments and apparatuses are all apparatuses or parts of apparatuses which are used in medicine, in surgery or in the hospital sector and are accessible on principle to mechanical cleaning.

The term “endoscopes and/or parts thereof” comprises all flexible and rigid endoscopes used in the field of diagnostics, therapy and surgery, which endoscopes can be soiled or contaminated during their intended use, and also parts of these instruments or apparatuses.

The inventive method is usable for rigid endoscopes (for example laparoscopes for minimally invasive surgery). This method is particularly advantageous in the case of flexible endoscopes, for example glass-fiber endoscopes, which, in their flexible regions, frequently have narrow-bore cavities which are difficult to clean. Endoscopes are flexible in the meaning of the invention if they have parts or regions which can bend or deform during their intended use.

The cleaning and disinfection are performed mechanically according to the invention. This means that intervention by hand during cleaning and/or disinfection is not required. In particular, the inventive method is suitable for use in customary washing machines for medical instruments or apparatuses in which the cleaning step and disinfection step can proceed in succession in the same washing tank. These washing machines preferably have devices or connections for the endoscopes so that the solution circulated in the machine in each case is also pumped through the channels of the endoscopes and cleans, disinfects or rinses these from the interior in this way.

The terms cleaning solution and disinfectant solution denote the ready-to-use corresponding aqueous solutions which are generally prepared by diluting concentrates with, preferably, softened or demineralized water.

According to the invention, during the disinfection step, chlorine dioxide is released from a chlorite in situ. This means that the chlorine dioxide is released in the aqueous disinfectant solution by a chemical reaction. In contrast, in the prior art, the chlorine dioxide is prepared (generally in a separate chlorine dioxide generator) by combining concentrated chlorite and acid solutions, what is termed this chloride dioxide stock solution is then diluted and not until then brought into contact with the parts to be disinfected. Getränkeindustrie 7/89, p. 600, describes, for example, a method familiar under the name Bellozon, in which the starting chemicals used for preparing chlorine dioxide are 9% strength hydrochloric acid and a 7.5% strength sodium chlorite solution. These chemicals are brought to reaction in a separate generator, and the resultant chlorine dioxide solution is then used as disinfectant.

The invention has recognized that this complex preparation avoids storing chlorine dioxide before the actual disinfection by reacting comparatively low concentrations of chlorite and acid in the finished disinfectant solution. According to the invention, a microbicidal chlorine dioxide concentration of 1 to 500 ppm, more preferably 2 to 200 ppm, more preferably 2 to 100 ppm, more preferably 5 to 100 ppm, more preferably 5 to 50 ppm, may easily be achieved in the disinfectant solution.

The chlorite concentration (calculated as sodium chlorite) in the disinfectant solution is preferably 0.01 to 1% by weight, more preferably 0.02 to 0.5% by weight, more preferably 0.05 to 0.3% by weight.

After adding the acid activator, generally a pH of 1 to 6, preferably 2 to 5, in particular about 2 to 4, is established. The cleaning solution is preferably not buffered in order to facilitate the pH change by adding the acid activator.

The concentration of acid (calculated as hydrochloric acid) required for the production of chlorine dioxide in situ can be, for example, 0.01 to 0.5% by weight, more preferably 0.02 to 0.2% by weight, more preferably 0.02 to 0.1% by weight. Higher amounts of acid for establishing the required pH for the chlorine dioxide release can be required, in particular, if the disinfectant solution contains buffers, for example phosphates. According to the invention, as acid activator, sulfuric acid or hydrochloric acid can be used, but generally less-corrosive acids, for example phosphoric acid, boric acid, or organic acids such as formic acid, acetic acid, oxalic acid, malonic acid, succinic acid, adipic acid, glycolic acid, lactic acid, gluconic acid, tartaric acid, malic acid, maleic acid, citric acid, ascorbic acid, amidosulfonic acid, sorbic acid or the like are used.

In the context of the invention, at the end of the cleaning step a), the aqueous cleaning solution can be discarded, and at the start of step b) an aqueous disinfectant solution can be freshly made up or metered in, in which then chlorine dioxide is prepared in situ by adding an acid activator.

However, in the context of the invention, it is particularly preferred if the aqueous cleaning solution used in step a) comprises chlorite from the start. After completion of the cleaning operation, this cleaning solution is then left as disinfectant solution in the washing machine and an acid activator is added thereto, so that the chlorine dioxide release begins in situ. If required, at the start of the disinfection step, chlorite-containing cleaner can be added once more before, during or after acid activator is added. This acid activator can be a relatively highly concentrated acid solution; when hydrochloric acid is used, a 14% strength HCl solution can be used, for example. In this manner the aqueous cleaning solution is used once more as disinfectant solution and, in addition, in the disinfection step is simultaneously sterilized in conjunction, so that after completion of the disinfection step, it can readily be discharged in the sewage system. By means of this method variant, the total time required for cleaning/disinfection can be considerably shortened.

The described variant of the inventive method is particularly advantageous because it can easily be used in conventional washing machines for cleaning and disinfecting medical instruments and apparatuses. Such washing machines generally have two metering pumps which are separate from one another, of which one is customarily used for metering a cleansing agent concentrate in the cleaning step, and the second is used for metering a disinfectant concentrate in the disinfection step. According to the invention, at the start of the cleaning step, a cleansing agent concentrate can be added by one of the pumps for preparing the cleaning solution; this concentrate then equally comprises chlorite, for example sodium chlorite or potassium chlorite, in the required amount.

After completion of the cleaning step, to initiate the disinfection step, merely an acid solution is added from the second metering pump as acid activator, so that in the used cleaning solution (which is now disinfectant solution) the release of chlorine dioxide begins in situ.

In the described method variant, the cleaner solution generally has a pH of 5 to 10, preferably about 6 to 9, and is thus in the neutral to slightly alkaline range. It can comprise customary cleaning constituents, such as surfactants, enzymes, complexing agents, phosphates, corrosion inhibitors etc.

The temperature during the disinfection can be between room temperature and close to the boiling temperature of the solutions used. In the cleaning of sensitive instruments, for example endoscopes or parts thereof, instruments from the field of anesthesia or the like, generally preference is given to temperatures of 20 to 60° C., in particular about 40° C.

A customary period for the cleaning step and disinfection step is in each case 1 minute to 1 hour, preferably about 2 to 30 minutes, more preferably about 2 to 15 minutes. Customary values are a period of 3 to 5 minutes for the cleaning step and 5 to 15 minutes, in particular about 5 to 10 minutes, for the disinfection step. The period of the disinfection step must be chosen to be sufficiently long that an effective microbicidal concentration can be established by the in-situ release of chlorine dioxide. The required duration of the disinfection step thus depends, in particular, also on the chlorite concentration and acid concentration used and on the temperature of the solution.

The listing of the inventive steps in the main claim is not final; customary prepurification steps, intermediate rinses and in particular final rinses can be provided. In particular, a final rinse with demineralized water is customary.

The cleaning and disinfectant solutions used inventively can be made up from concentrates using softened or demineralized water, but they can also be made up using customary municipal water.

An exemplary embodiment of the invention is described hereinafter.

A cleaner concentrate (component A) and an acid activator concentrate (component B) are made up as follows:

Component A:

10% sodium chlorite solution, 30% strength

40% potassium tripolyphosphate solution, 50% strength

50% demineralized water

Component B:

12% strength hydrochloric acid

In a customary washing machine for medical instruments, foul instruments were cleaned for a period of 15 min using a 1% strength aqueous solution of component A at 50° C.

After completion of the cleaning operation, the disinfection operation was initiated by adding 1% strength by weight (based on the cleaning bath) of the acid activator component B. As a result of this addition, the cleaning solution was converted into a disinfectant solution within the meaning of the invention. After the addition, the pH of the disinfectant solution decreased to about 2 and chlorine dioxide formation started. The disinfection was carried out over a period of 10 min at a temperature of 50° C. After this period, a chlorine dioxide content of about 20 ppm had been established in the disinfectant solution.

After completion of the disinfection step, the sterile disinfectant solution was drained off and the instruments were further rinsed with demineralized water.

1. A method for mechanically cleaning and disinfecting medical and surgical instruments and apparatuses having the following steps:
a) cleaning the instruments and apparatuses with an aqueous cleaning solution,
b) disinfecting the instruments and apparatuses with an aqueous disinfectant solution,
characterized in that, during the disinfection step, as disinfectant, chlorine dioxide is released in situ from chlorite and an acid activator.

2. The method as claimed in claim 1, characterized in that the chlorite concentration (calculated as sodium chlorite) in the disinfectant solution is preferably 0.01 to 1% by weight, more preferably 0.02 to 0.5% by weight, more preferably 0.05 to 0.3% by weight.

3. The method as claimed in claim 1 or 2, characterized in that, in the disinfection step, after adding the acid activator, a pH of 1 to 6, preferably 2 to 5, more preferably 2 to 4, is established.

4. The method as claimed in claims 1 to 3, characterized in that, in the disinfectant solution, a chlorine dioxide concentration is achieved of 1 to 500 ppm, preferably 2 to 200 ppm, more preferably 2 to 100 ppm, more preferably 5 to 100 ppm, more preferably 5 to 50 ppm.

5. The method as claimed in one of claims 1 to 4, characterized in that, in step b), a chlorite-containing solution and an acid activator are added separately.

6. The method as claimed in one of claims 1 to 4, characterized in that the aqueous cleaning solution added in step a) comprises chlorite and, in step b), an acid activator is added.

7. The method as claimed in one of claims 1 to 6, characterized in that the period of the disinfection step is 1 min to 1 hour, preferably 2 to 30 min, more preferably 2 to 15 min, more preferably 5 to 10 min.

8. The method as claimed in one of claims 1 to 7, the medical instrument or the medical apparatus being an endoscope and/or parts thereof.

9. A kit for carrying out the method as claimed in one of claims 1 to 8, characterized in that it contains:
a) a cleaner concentrate,
b) a disinfectant concentrate which comprises chlorite,
c) an acid activator concentrate.

10. A kit for carrying out the method as claimed in one of claims 1 to 8, characterized in that it comprises:
a) a cleaner concentrate, which comprises chlorite,
b) an acid activator concentrate.
